(19) United States
(12) Patent Application Publication
(10) Pub. No.: US 2025/0144102 A1
(43) Pub. Date: May 8, 2025

(54) 1 H-PYRAZOLO[4,3-D]PYRIMIDINE DERIVATIVES AS STAPHYLOCOCCUS AUREUS INHIBITORS

(71) Applicant: DR REDDY'S INSTITUTE OF LIFE SCIENCES, Hyderabad, Telangana (IN)

(72) Inventors: Srinivas ORUGANTI, Hyderabad (IN); Parimal MISRA, Hyderabad (IN); Nasreen Zafar EHTESHAM, New Delhi (IN); Seyed Ehtesham HASNAIN, New Delhi (IN); Manojit PAL, Hyderabad (IN)

(73) Assignee: DR REDDY'S INSTITUTE OF LIFE SCIENCES, Hyderabad, Telangana (IN)

(21) Appl. No.: 18/834,944

(22) PCT Filed: Feb. 1, 2023
(86) PCT No.: PCT/IN2023/050098

§ 371 Date: Jul. 31, 2024

(30) Foreign Application Priority Data
Feb. 1, 2022 (IN) 202241005390

(51) Int. Cl. A61K 31/519; A61P 31/04

(52) U.S. Cl. CPC A61K 31/519; A61P 31/04

(57) ABSTRACT

The present invention relates to a compound of Formula (I): [Formula should be inserted here] or a pharmaceutically acceptable salt, a pharmaceutically acceptable stereoisomer, a pharmaceutically acceptable hydrate, a pharmaceutically acceptable solvate, a prodrug, a polymorph, an N-oxide or a bioisostere thereof; wherein “R”, “Ar”, and “Het” are as defined in the specification, which can be used as a medicament or therapeutic agent in the prevention or treatment of a condition or a disease or a disorder where there is an advantage in inhibiting S. aureus or other Gram-positive bacteria.

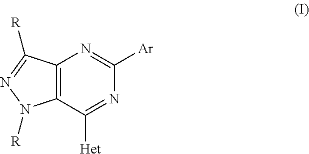

### CROSS-REFERENCE TO RELATED APPLICATIONS

This application claims the benefit of Indian Provisional Patent Application No. 202241005390, filed Feb. 1, 2022, which is incorporated by reference herein in its entirety.

### FIELD OF THE INVENTION

The present invention relates to Staphylococcus aureus (S. aureus) inhibitors, more particularly to 1H-pyrazolo[4,3-d]pyrimidine derivatives as S. aureus inhibitors.

### BACKGROUND OF THE INVENTION

Staphylococcus aureus is a human pathogen that can cause a range of illnesses, from minor skin infections to life-threatening diseases, such as pneumonia, meningitis, endocarditis, toxic shock syndrome (TSS), bacteraemia, and sepsis. It is one of the most common causes of nosocomial infections and is often the cause of postsurgical wound infections. Methicillin-resistant S. aureus (MRSA) strains, which have become resistant to most antibiotics, are most often found associated with institutions such as hospitals, but they are also becoming increasingly prevalent in community-acquired infections. The danger of MRSA infections results from not only the emergence of multidrug resistance but also the occurrence of strong biofilm-forming bacteria.

Thus, there is a need to provide therapeutic compounds, compositions and/or methods for inhibiting Staphylococcus aureus.

### SUMMARY OF THE INVENTION

Provided herein is an inhibitor of S. aureus, particularly a compound of Formula (I):

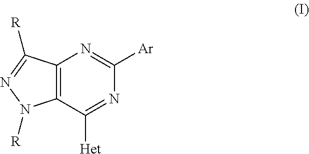

or a pharmaceutically acceptable salt, a pharmaceutically acceptable stereoisomer, a pharmaceutically acceptable hydrate, a pharmaceutically acceptable solvate, a prodrug, a polymorph, an N-oxide or a bioisostere thereof, as S. aureus inhibitor; wherein:
each R is independently selected from hydrogen, halogen, hydroxyl, optionally substituted alkyl, optionally substituted alkoxy, optionally substituted cycloalkyl, optionally substituted aryl, optionally substituted heterocyclyl, and optionally substituted heteroaryl;
Ar is an aryl or heteroaryl; and
Het represents an optionally substituted heterocyclyl or heteroaryl connected through a C—C bond.

In an aspect, the compound Formula (I) is for use as a medicament or therapeutic agent in the prevention or treatment of a condition or a disease or a disorder caused by S. aureus.

In another aspect, the present invention provides a pharmaceutical composition comprising at least one compound of Formula (I), and its use for the treatment or prevention in a condition or a disease or a disorder, in particular their use in a condition or a disease or a disorder where there is an advantage in inhibiting S. aureus or other Gram-positive bacteria.

In yet another aspect, the present invention provides a method of preventing or treating a condition or a disease or a disorder caused by S. aureus in a subject. The method comprises administering to the subject a therapeutically effective amount of a compound of Formula (I) described herein.

In a further aspect, the present invention provides a method of inhibiting S. aureus; wherein the method comprises administering to a subject in a need thereof a therapeutically effective amount of a compound of Formula (I) described herein.

In another aspect, the present invention provides a compound for use in preventing or treating a condition or a disease or a disorder caused by S. aureus in a subject, wherein the compound is a compound of Formula (I) described herein.

In yet another aspect, the present invention provides a compound for use in inhibiting S. aureus; wherein the compound is a compound of Formula (I) described herein.

### DETAILED DESCRIPTION OF THE INVENTION

Unless defined otherwise, all technical and scientific terms used herein have the same meaning as commonly understood by those of ordinary skill in the art to which the invention belongs. Although any methods and materials similar or equivalent to those described herein can be used in the practice or testing of the present invention, preferred methods and materials are described. For the purposes of the present invention, the following terms are defined below.

The articles “a” and “an” are used herein to refer to one or to more than one (i.e. to at least one) of the grammatical object of the article. By way of example, “an element” means one element or more than one element.

The term “alkyl” refers to a straight or branched chain saturated aliphatic hydrocarbon that may be substituted or unsubstituted. In certain embodiments, the alkyl is C₁-C₆ alkyl. Examples of “alkyl” include but are not limited to methyl, ethyl, n-propyl, isopropyl, n-butyl, t-butyl, n-pentyl, isobutyl and the likes thereof.

The term “alkoxy” refers to a group —O-alkyl, wherein alkyl is as defined above. Representative examples include, but are not limited to, methoxy, ethoxy, propoxy, t-butoxy and the likes thereof.

The term “aryl” refers to optionally substituted unsaturated or partially saturated aromatic ring system having five to ten carbon atoms which are monocyclic, bicyclic, or polycyclic and may optionally be replaced by one or more hetero atoms selected from N, O and S. Exemplary aryl groups include phenyl, naphthyl, indanyl, biphenyl and the likes thereof.

The term “bioisostere” refers to a compound or group that possesses near molecular shapes and volumes, approximately the same distribution of electrons and which exhibit similar physical properties such as hydrophobicity. Bioisostereic compounds affect the same biochemically associated systems as agonist or antagonists and thereby produce biological properties that are related to each other.

As used herein, the term ‘compound(s)’ comprises the compounds disclosed in the present invention.

As used herein, the term “comprises” or “comprising” is generally used in the sense of include, that is to say permitting the presence of one or more features or components.

As used herein, the term “composition” is intended to encompass a product comprising the specified ingredients in the specified amounts, as well as any product which results, directly or indirectly, from combination of the specified ingredients in the specified amounts. By “pharmaceutically acceptable” it is meant the carrier, diluent or excipient must be compatible with the other ingredients of the formulation and not deleterious to the recipient thereof.

“Compounds of the invention” or “present invention” refers to the compounds of Formula (I) as herein defined, a pharmaceutically acceptable salt, a pharmaceutically acceptable stereoisomer, a pharmaceutically acceptable hydrate, a pharmaceutically acceptable solvate, a prodrug, a polymorph, an N-oxide or a bioisostere thereof and a pharmaceutically acceptable composition containing them. The compounds of the present invention may be useful as antibacterial agents particularly in the treatment of a disease or disorder where there is an advantage in inhibiting S. or other Gram-positive bacteria.

As used herein, the term “cycloalkyl” used herein, either alone or in combination with other radicals, denotes mono, bicyclic, or polycyclic saturated, partially saturated hydrocarbon ring system of about 3 to 12 carbon atoms which may be substituted or unsubstituted. Exemplary “cycloalkyl” groups include but are not limited to cyclopopyl, cyclobutyl, cyclopentyl, cyclohexyl and cycloheptyl, perhydronapthyl, adamantyl, noradamantyl and spirobicyclic groups such as spiro (4,4)non-2-yl.

The term “halogen” or “halo” refers to fluorine, chlorine, bromine, or iodine.

As used herein, the term “heteroaryl” refers to monocyclic aromatic ring systems or fused bicyclic aromatic ring systems comprising two or more aromatic rings, preferably two to three ring systems. These heteroaryl rings contain one or more nitrogen, sulfur and/or oxygen atoms where N-oxides sulfur oxides and dioxides are permissible heteroatom substitutions. The term includes ring(s) optionally substituted with halogens, nitro, amino, alkoxy, alkyl sulfonyl amino, alkylcarbonylamino, carboxy, alkyl carbonoyl, hydroxy, and alkyl. Examples of heteroaryl groups include furan, thiophene, pyrrole, imidazole, pyrazole, triazole, tetrazole, thiazole, oxazole, isoxazole, oxadiazole, thiadiazole, isothiazole, pyridine, pyridazine, pyrazine, pyrimidine, quinoline, isoquinoline, benzofuran, benzothiophene, indole, indazole, chromanyl, isochromanyl and the likes thereof.

The term “heterocyclyl” refers to a stable 3 to 15 membered ring that is either saturated or has one or more degrees of unsaturation or unsaturated. These heterocyclic rings contain one or more heteroatoms selected from the group consisting of nitrogen, sulfur, and oxygen where N-oxides, sulfur oxides and dioxides are permissible heteroatom substitutions. Such a ring is optionally fused to one or more of another heterocyclic ring(s), aryl ring(s) or cycloalkyl ring(s). Examples of such groups are selected from the group consisting of azetidinyl, acridinyl, pyrazolyl, imidazolyl, triazolyl, pyrrolyl, thiophenyl, thiazolyl, oxazolyl, isoxazolyl, furanyl, pyrazinyl, tetrahydroisoquinolinyl, piperidinyl, piperazinyl, morpholinyl, thiomorphonilyl, pyridazinyl, indolyl, isoindolyl, quinolinyl, chromanyl and the likes thereof. “Heterocyclylalkyl” refers to a heterocyclic ring radical defined above, directly bonded to an alkyl group. The heterocyclylalkyl radical is attached to the main structure at carbon atom in the alkyl group that results in the creation of a stable structure.

“N-oxide” refers to a nitrogen atom oxide of a nitrogen-containing heteroaryl or heterocyclyl. The N-oxide can be formed in the presence of an oxidizing agent such as, for example, a peroxide of meta-chloro-perbenzoic acid or hydrogen peroxide. N-oxide means a compound containing an N→O bond.

The term “hydroxyl” refers to —OH group.

As used herein, the terms “optional” or “optionally” mean that the subsequently described event or circumstance may occur or may not occur, and that the description includes instances where the event or circumstance occurs as well as instances in which it does not. For example, “optionally substituted alkyl” refers to the alkyl may be substituted as well as the event or circumstance where the alkyl is not substituted.

“Pharmaceutically acceptable” means that, which is useful in preparing a pharmaceutical composition that is generally safe, non-toxic and neither biologically nor otherwise undesirable and includes that which is acceptable for veterinary as well as human pharmaceutical use.

“Pharmaceutically acceptable salt” refers to the salts of the compounds, that is pharmaceutically acceptable and that possesses the desired pharmacological activity of the parent compound. Pharmaceutically acceptable salts of the compounds of this invention include those derived from suitable inorganic and organic acids and bases. Such salts include: salts derived from inorganic bases such as Li, Na, K, Ca, Mg, Fe, Cu, Zn, Al, Mn; salts of organic bases such as N,N′-diacetylethylenediamine, 2-dimethylaminoethanol, isopropylamine, morpholine, piperazine, piperidine, procaine, diethylamine, triethylamine, trimethylamine, tripropylamine, tromethamine, choline hydroxide, dicyclohexylamine, metformin, benzylamine, phenylethylamine, dialkylamine, trialkylamine, thiamine, aminopyrimidine, aminopyridine, purine, pyrimidine, spermidine, and the like; chiral bases like alkylphenylamine, glycinol, phenyl glycinol and the like, salts of natural amino acids such as glycine, alanine, valine, leucine, isoleucine, lysine, arginine, serine, threonine, phenylalanine; unnatural amino acids such as D-isomers or substituted amino acids; salts of acidic amino acids such as aspartic acid, glutamic acid; guanidine, substituted guanidine wherein the substituents are selected from nitro, amino, alkyl, alkenyl, alkynyl, ammonium or substituted ammonium salts. Salts may include acid addition salts where appropriate which are sulphates, nitrates, phosphates, perchlorates, borates, hydrohalides, acetates, tartrates, maleates, citrates, succinates, methanesulfonates, benzoates, salicylates, hydroxynaphthoates, benzenesulfonates, ascorbates, and the likes thereof.

The term “polymorph” refers to the crystallization of the same compound, which differs only in the lattice.

As used herein, the term “prevents”, “preventing” and “prevention” refer to a method of preventing the onset of a disease and/or its attendant symptoms or barring a subject from acquiring a disease. As used herein, “prevent”, “preventing” and “prevention” also include delaying the onset of a disease and/or its attendant symptoms and reducing a subject's risk of acquiring a disease.

The term “solvate” or “solvates” refers to a complex in which the compounds of the Formula (I) of the present invention are a proportional amount of solvent molecules combined. A specific solvate in which the solvent is water is referred to as a hydrate.

Unless otherwise specified, the term “substituted” as used herein refers to mono, bi, tri or tetra substitution with any one or combination of the following substituents: hydroxy, halogen, carboxyl, cyano, nitro, oxo (═O), thio (═S), substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted or unsubstituted heterocyclylalkyl ring, substituted or unsubstituted heteroarylalkyl, substituted or unsubstituted heterocyclic ring, substituted or unsubstituted guanidine, —COOR², —C(O)R², —C(S)R², —C(O)NR²R³, —C(O)ONR³R⁴, —NR²CONR³R⁴, —N(R²)SOR³, —N(R²)SO₂R³, —(═N—N(R²)R³), —NR²C(O)OR³, —NR²R³, —NR²C(O)R³, —NR²C(S)R³, —NR²C(S)NR³R⁴, —SONR²R³, —SO₂NR²R³, —OR², —OR²C(O)NR³R⁴, —OR²C(O)OR³, —OC(O)R², —OC(O)NR²R³, —R²NR³C(O)R⁴, —R²OR³, —R²C(O)OR³, —R²C(O)NR³R⁴, —R²C(O)R³, —R²OC(O)R², —SR², —SOR², —SO₂R², and —ONO², wherein R², R³ and R⁴ are independently selected from hydrogen, substituted or unsubstituted alkyl, substituted or unsubstituted alkoxy, substituted or unsubstituted alkenyl, substituted or unsubstituted alkynyl, substituted or unsubstituted aryl, substituted or unsubstituted arylalkyl, substituted or unsubstituted cycloalkyl, substituted or unsubstituted cycloalkenyl, substituted or unsubstituted amino, substituted or unsubstituted aryl, substituted or unsubstituted heteroaryl, substituted heterocyclylalkyl ring, substituted or unsubstituted heteroarylalkyl, or substituted or unsubstituted heterocyclic ring. Alternately R³ and R⁴ together along with the nitrogen they are attached with, form a 4 to 8 membered ring which can be substituted or unsubstituted. According to one embodiment, the substituents in the aforementioned “substituted” groups cannot be further substituted. For example, when the substituent on “substituted alkyl” is “substituted aryl” the substituent on “substituted aryl” cannot be “substituted alkenyl”.

The term “stereoisomer” or “stereoisomers” refers to any enantiomers, diastereomers or geometrical isomers of the compounds of Formula (I), wherever they are chiral or when they bear one or more double bond. When the compounds of the Formula (I) and related formulae are chiral, they can exist in racemic or in optically active form. It should be understood that the invention encompasses all stereochemical isomeric forms, including diastereomeric, enantiomeric and epimeric forms, as well as -isomers and/-isomers and mixtures thereof. Individual stereoisomers of compounds can be prepared synthetically from commercially available starting materials which contain chiral centers or by preparation of mixtures of enantiomeric products followed by separation such as conversion to a mixture of diastereomers followed by separation or recrystallization, chromatographic techniques, direct separation of enantiomers on chiral chromatographic columns, or any other appropriate method known in the art. Starting compounds of particular stereochemistry are either commercially available or can be made and resolved by techniques known in the art. Additionally, the compounds of the present invention may exist as geometric isomers. The present invention includes all cis, trans, syn, anti, entgegen (E) and zusammen (Z) isomers as well as the appropriate mixtures thereof.

The stereoisomers of the compounds forming part of this invention may be prepared by using reactants in their single enantiomeric form in the process wherever possible or by conducting the reaction in the presence of reagents or catalysts in their single enantiomer form or by resolving the mixture of stereoisomers by conventional methods. Some of the preferred methods include use of microbial resolution, resolving the diastereomeric salts formed with chiral acids such as mandelic acid, camphorsulfonic acid, tartaric acid, lactic acid, and the like wherever applicable or chiral bases such as brucine, cinchona alkaloids and their derivatives and the like. Different polymorphs of a compound of Formula (I) of present invention may be prepared by crystallization of the corresponding compound of Formula (I) under different conditions. For example, making use of commonly used solvents or their mixtures for recrystallization, crystallization at different temperature ranges, different cooling techniques like very fast to very slow cooling during crystallization procedure, by exposing to room temp, by heating or melting the compound followed by gradual cooling and the like. The presence of polymorphs may be determined by one or more methods like solid probe NMR spectroscopy, DSC, TGA, Powder X-Ray diffraction and IR.

As used herein, the term “therapeutically effective amount” refers to that amount of the compound being administered sufficient to prevent development of or alleviate to some extent one or more of the symptoms of the condition or disorder being treated.

As used herein, the term “treat”, “treating” and “treatment” refer to a method of alleviating or abrogating a disease and/or its attendant symptoms.

Each embodiment is provided by way of explanation of the invention and not by way of limitation of the invention. In fact, it will be apparent to those skilled in the art that various modifications and variations can be made to the compounds, compositions and methods described herein without departing from the scope or spirit of the invention. For instance, features illustrated or described as part of one embodiment can be applied to another embodiment to yield a still further embodiment. Thus, it is intended that the present invention includes such modifications and variations and their equivalents. Other objects, features and aspects of the present invention are disclosed in or are obvious from, the following detailed description. It is to be understood by one of ordinary skill in the art that the present discussion is a description of exemplary embodiments only and is not to be construed as limiting the broader aspects of the present invention.

In an embodiment, the present disclosure provides a compound of Formula (I):

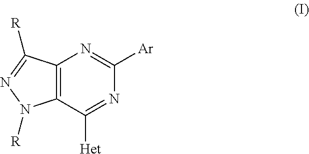

or a pharmaceutically acceptable salt, a pharmaceutically acceptable stereoisomer, a pharmaceutically acceptable hydrate, a pharmaceutically acceptable solvate, a prodrug, a polymorph, an N-oxide or a bioisostere thereof, as S. aureus inhibitor; wherein,
each R is independently selected from hydrogen, halogen, hydroxyl, optionally substituted alkyl, optionally substituted alkoxy, optionally substituted cycloalkyl, optionally substituted aryl, optionally substituted heterocyclyl, and optionally substituted heteroaryl;
Ar is an aryl or heteroaryl; and
Het represents an optionally substituted heterocyclyl or heteroaryl group connected through a C—C bond.

In certain embodiments, R is optionally substituted alkyl. In some instances, R is alkyl. In further instances, R is C₁-C₆ alkyl. In further embodiments, R is methyl, ethyl, or propyl.

In certain embodiments, Ar is aryl. In some instances, Ar is phenyl optionally substituted with one or more halo. In further instances, Ar is phenyl substituted with one or more groups independently selected from chloro and bromo.

In certain embodiments, Het is an optionally substituted heteroaryl. In further embodiments, Het is an optionally substituted indole group connected to the pyrimidine moiety through a C—C bond.

In certain embodiments, Het is indole substituted with one or more groups independently selected from halo and alkyl. In some instances, Het is indole substituted with one or more groups independently selected from bromo, chloro, methyl and ethyl.

In certain embodiments, Het is selected from the following:

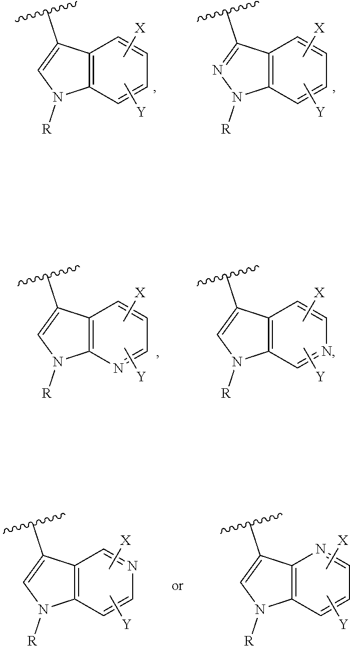

wherein,
R is independently selected from the group consisting of hydrogen, halogen, hydroxyl, optionally substituted alkyl, optionally substituted alkoxy, optionally substituted cycloalkyl, optionally substituted aryl, optionally substituted heterocyclyl and optionally substituted heteroaryl;
X is hydrogen or halo; and
Y is hydrogen or halo group.

In certain embodiments, the compounds of the present disclosure can also contain unnatural proportions of atomic isotopes at one or more of the atoms that constitute such compounds. For example, the present invention also embraces isotopically-labeled variants of the present invention which are identical to those recited herein, but for the fact that one or more atoms of the compound are replaced by an atom having the atomic mass or mass number different from the predominant atomic mass or mass number usually found in nature for the atom. All isotopes of any particular atom or element as specified are contemplated within the scope of the compounds of the invention and their uses. Exemplary isotopes that can be incorporated in to compounds of the invention include isotopes of hydrogen, carbon, nitrogen, oxygen, phosphorous, sulfur, fluorine, chlorine and iodine, such as ²H (“D”), ³H, ⁿC, ¹³C, ¹⁴C, ¹³N, ¹⁵N, ¹⁵O, ¹⁷O, ¹⁸O, ³⁵S, ¹⁸F, ³⁶Cl, ¹²³I and ¹²⁵I. Isotopically labeled compounds of the present inventions can generally be prepared by following procedures analogous to those disclosed in the schemes and/or in the examples herein below, by substituting an isotopically labeled reagent for a non-isotopically labeled reagent.

In certain embodiments, the present disclosure provides a compound selected from:
5-(4-chlorophenyl)-7-(1H-indol-3-yl)-1-methyl-3-propyl-1H-pyrazolo[4,3-d]pyrimidine;
5-(4-chlorophenyl)-1-methyl-7-(1-methyl-1H-indol-3-yl)-3-propyl-1H-pyrazolo[4,3-d]pyrimidine;
5-(4-chlorophenyl)-7-(1-ethyl-1H-indol-3-yl)-1-methyl-3-propyl-1H-pyrazolo[4,3-d]pyrimidine;
7-(5-bromo-1H-indol-3-yl)-5-(4-chlorophenyl)-1-methyl-3-propyl-1H-pyrazolo[4,3-d]pyrimidine;
7-(5-bromo-1-methyl-1H-indol-3-yl)-5-(4-chlorophenyl)-1-methyl-3-propyl-1H-pyrazolo[4,3-d]pyrimidine;
7-(5-bromo-1-ethyl-1H-indol-3-yl)-5-(4-chlorophenyl)-1-methyl-3-propyl-1H-pyrazolo[4,3-d]pyrimidine;
7-(5-chloro-1H-indol-3-yl)-5-(4-chlorophenyl)-1-methyl-3-propyl-1H-pyrazolo[4,3-d]pyrimidine;
7-(5-chloro-6-fluoro-1H-indol-3-yl)-5-(4-chlorophenyl)-1-methyl-3-propyl-1H-pyrazolo[4,3-d]pyrimidine;
5-(4-chlorophenyl)-7-(5,6-difluoro-1H-indol-3-yl)-1-methyl-3-propyl-1H-pyrazolo[4,3-d]pyrimidine;
5-(4-chlorophenyl)-7-(5-methoxy-1H-indol-3-yl)-1-methyl-3-propyl-1H-pyrazolo[4,3-d]pyrimidine;
4-chlorophenyl)-7-(5-methoxy-1-methyl-1H-indol-3-yl)-1-methyl-3-propyl-1H-pyrazolo[4,3-d]pyrimidine;
5-(4-chlorophenyl)-7-(1-ethyl-5-methoxy-1H-indol-3-yl)-1-methyl-3-propyl-1H-pyrazolo[4,3-d]pyrimidine; and
7-(1-allyl-1H-indol-3-yl)-5-(4-chlorophenyl)-1-methyl-3-propyl-1H-pyrazolo[4,3-d]pyrimidine;
   or a pharmaceutically acceptable salt, a pharmaceutically acceptable stereoisomer, a pharmaceutically acceptable hydrate, a pharmaceutically acceptable solvate, a prodrug, a polymorph, an N-oxide or a bioisostere thereof.

In certain embodiments, the present disclosure provides a pharmaceutical composition comprising a compound of Formula (I) or a pharmaceutically acceptable salt, a pharmaceutically acceptable stereoisomer, a pharmaceutically acceptable hydrate, a pharmaceutically acceptable solvate, a prodrug, a polymorph, an N-oxide or a bioisostere thereof, optionally admixed with a pharmaceutically acceptable carrier or excipient or diluent.

The present disclosure also provides methods for formulating the disclosed compounds for pharmaceutical administration.

The compositions and methods of the present invention may be utilized to treat a subject in need thereof. In certain embodiments, the subject is a mammal such as a human or a non-human mammal. When administered to an animal, such as a human, the composition or the compound is preferably administered as a pharmaceutical composition comprising, for example, a compound of the invention and a pharmaceutically acceptable carrier. Pharmaceutically acceptable carriers are well known in the art and include, for example, aqueous solutions such as water or physiologically buffered saline or other solvents or vehicles such as glycols, glycerol, and oils such as olive oil or injectable organic esters. In a preferred embodiment, when such pharmaceutical compositions are for human administration, particularly for invasive routes of administration (i.e., routes, such as injection or implantation, that circumvent transport or diffusion through an epithelial barrier), the aqueous solution is pyrogen-free or substantially pyrogen-free. The excipients can be chosen, for example, to effect delayed release of an agent or to selectively target one or more cells, tissues, or organs. The pharmaceutical composition can be in dosage unit form such as tablet, capsule (including sprinkle capsule and gelatin capsule), granule, and lyophile for reconstitution, powder, solution, syrup, suppository, injection, or the like. The composition can also be present in a transdermal delivery system, e.g., a skin patch. The composition can also be present in a solution suitable for topical administration, such as an eye drop.

A pharmaceutically acceptable carrier can contain physiologically acceptable agents that act, for example, to stabilize, increase solubility or to increase the absorption of a compound such as a compound of the invention. Such physiologically acceptable agents include, for example, carbohydrates, such as glucose, sucrose or dextrans, antioxidants, such as ascorbic acid or glutathione, chelating agents, low molecular weight proteins or other stabilizers or excipients. The choice of a pharmaceutically acceptable carrier, including a physiologically acceptable agent, depends, for example, on the route of administration of the composition. The preparation of pharmaceutical composition can be a self-emulsifying drug delivery system or a self-microemulsifying drug delivery system. The pharmaceutical composition (preparation) also can be a liposome or other polymer matrix, which can have incorporated therein, for example, a compound of the invention. Liposomes, for example, which comprise phospholipids or other lipids, are nontoxic, physiologically acceptable, and metabolizable carriers that are relatively simple to make and administer.

The phrase “pharmaceutically acceptable” is employed herein to refer to those compounds, materials, compositions and/or dosage forms which are, within the scope of sound medical judgment, suitable for use in contact with the tissues of human beings and animals without excessive toxicity, irritation, allergic response or other problem or complication, commensurate with a reasonable benefit/risk ratio.

The phrase “pharmaceutically acceptable carrier” as used herein means a pharmaceutically acceptable material, composition, or vehicle, such as a liquid or solid filler, diluent, excipient, solvent, or encapsulating material. Each carrier must be “acceptable” in the sense of being compatible with the other ingredients of the formulation and not injurious to the patient. Some examples of materials which can serve as pharmaceutically acceptable carriers include: (1) sugars, such as lactose, glucose and sucrose; (2) starches, such as corn starch and potato starch; (3) cellulose and its derivatives, such as sodium carboxymethyl cellulose, ethyl cellulose and cellulose acetate; (4) powdered tragacanth; (5) malt; (6) gelatin; (7) talc; (8) excipients, such as cocoa butter and suppository waxes; (9) oils, such as peanut oil, cottonseed oil, safflower oil, sesame oil, olive oil, corn oil and soybean oil; (10) glycols, such as propylene glycol; (11) polyols, such as glycerin, sorbitol, mannitol and polyethylene glycol; (12) esters, such as ethyl oleate and ethyl laurate; (13) agar; (14) buffering agents, such as magnesium hydroxide and aluminum hydroxide; (15) alginic acid; (16) pyrogen-free water; (17) isotonic saline; (18) Ringer's solution; (19) ethyl alcohol; (20) phosphate buffer solutions; and (21) other non-toxic compatible substances employed in pharmaceutical formulations.

A pharmaceutical composition (preparation) can be administered to a subject by any of a number of routes of administration including, for example orally (for example, drenches as in aqueous or non-aqueous solutions or suspensions, tablets, capsules (including sprinkle capsules and gelatin capsules), boluses, powders, granules, pastes for application to the tongue); absorption through the oral mucosa (e.g., sublingually); anally, rectally or vaginally (for example, as a pessary, cream or foam); parenterally (including intramuscularly, intravenously, subcutaneously or intrathecally as, for example, a sterile solution or suspension); nasally; intraperitoneally; subcutaneously; transdermally (for example as a patch applied to the skin); and topically (for example, as a cream, ointment or spray applied to the skin or as an eye drop). The compound may also be formulated for inhalation. In yet another embodiment, a compound may be simply dissolved or suspended in sterile water.

The formulations may conveniently be presented in unit dosage form and may be prepared by any methods well known in the art of pharmacy. The amount of active ingredient which can be combined with a carrier material to produce a single dosage form will vary depending upon the host being treated, and the particular mode of administration. The amount of active ingredient that can be combined with a carrier material to produce a single dosage form will generally be that amount of the compound which produces a therapeutic effect. Generally, out of one hundred percent, this amount will range from about 1 percent to about ninety-nine percent of active ingredient, preferably from about 5 percent to about 70 percent, most preferably from about 10 percent to about 30 percent.

Methods of preparing these formulations or compositions include the step of bringing into association an active compound, such as a compound of the invention, with the carrier and, optionally, one or more accessory ingredients. In general, the formulations are prepared by uniformly and intimately bringing into association a compound of the present invention with liquid carriers or finely divided solid carriers or both and then, if necessary, shaping the product.

Formulations of the invention suitable for oral administration may be in the form of capsules (including sprinkle capsules and gelatin capsules), cachets, pills, tablets, lozenges (using a flavored basis, usually sucrose and acacia or tragacanth), lyophile, powders, granules or as a solution or a suspension in an aqueous or non-aqueous liquid or as an oil-in-water or water-in-oil liquid emulsion or as an elixir or syrup or as pastilles (using an inert base, such as gelatin and glycerin or sucrose and acacia) and/or as mouth washes and the like, each containing a predetermined amount of a compound of the present invention as an active ingredient. Compositions or compounds may also be administered as a bolus, electuary or paste.

To prepare solid dosage forms for oral administration (capsules (including sprinkle capsules and gelatin capsules), tablets, pills, dragees, powders, granules and the like), the active ingredient is mixed with one or more pharmaceutically acceptable carriers, such as sodium citrate or dicalcium phosphate and/or any of the following: (1) fillers or extenders, such as starches, lactose, sucrose, glucose, mannitol and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates and sodium carbonate; (5) solution retarding agents, such as paraffin; (6) absorption accelerators, such as quaternary ammonium compounds; (7) wetting agents, such as, for example, cetyl alcohol and glycerol monostearate; (8) absorbents, such as kaolin and bentonite clay; (9) lubricants, such a talc, calcium stearate, magnesium stearate, solid polyethylene glycols, sodium lauryl sulfate and mixtures thereof; (10) complexing agents, such as, modified and unmodified cyclodextrins; and (11) coloring agents. In the case of capsules (including sprinkle capsules and gelatin capsules), tablets and pills, the pharmaceutical compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the powdered compound moistened with an inert liquid diluent.

The tablets and other solid dosage forms of the pharmaceutical compositions, such as dragees, capsules (including sprinkle capsules and gelatin capsules), pills and granules, may optionally be scored or prepared with coatings and shells, such as enteric coatings and other coatings well known in the pharmaceutical-formulating art. They may also be formulated so as to provide slow or controlled release of the active ingredient therein using, for example, hydroxypropylmethyl cellulose in varying proportions to provide the desired release profile, other polymer matrices, liposomes and/or microspheres. They may be sterilized by, for example, filtration through a bacteria-retaining filter or by incorporating sterilizing agents in the form of sterile solid compositions that can be dissolved in sterile water or some other sterile injectable medium immediately before use. These compositions may also optionally contain opacifying agents and may be of a composition that they release the active ingredient(s) only or preferentially, in a certain portion of the gastrointestinal tract, optionally, in a delayed manner. Examples of embedding compositions that can be used include polymeric substances and waxes. The active ingredient can also be in microencapsulated form, if appropriate, with one or more of the above-described excipients.

Liquid dosage forms useful for oral administration include pharmaceutically acceptable emulsions, lyophiles for reconstitution, microemulsions, suspensions, solutions, syrups, and elixirs. In addition to the active ingredient, the liquid dosage forms may contain inert diluents commonly used in the art, such as, for example, water or other solvents, cyclodextrins and derivatives thereof, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan and mixtures thereof.

Besides inert diluents, the oral compositions can also include adjuvants such as wetting agents, emulsifying and suspending agents, sweetening, flavoring, coloring, perfuming and preservative agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth and mixtures thereof.

Formulations of the pharmaceutical compositions for rectal, vaginal or urethral administration may be presented as a suppository, which may be prepared by mixing one or more active compounds with one or more suitable nonirritating excipients or carriers comprising, for example, cocoa butter, polyethylene glycol, a suppository wax or a salicylate and which is solid at room temperature, but liquid at body temperature and, therefore, will melt in the rectum or vaginal cavity and release the active compound.

Formulations of the pharmaceutical compositions for administration to the mouth may be presented as a mouthwash or an oral spray or an oral ointment.

Alternatively, or additionally, compositions can be formulated for delivery via a catheter, stent, wire, or other intraluminal device. Delivery via such devices may be especially useful for delivery to the bladder, urethra, ureter, rectum, or intestine.

Formulations which are suitable for vaginal administration also include pessaries, tampons, creams, gels, pastes, foams, or spray formulations containing such carriers as are known in the art to be appropriate.

Dosage forms for the topical or transdermal administration include powders, sprays, ointments, pastes, creams, lotions, gels, solutions, patches, and inhalants. The active compound may be mixed under sterile conditions with a pharmaceutically acceptable carrier and with any preservatives, buffers or propellants that may be required.

The ointments, pastes, creams, and gels may contain, in addition to an active compound, excipients, such as animal and vegetable fats, oils, waxes, paraffins, starch, tragacanth, cellulose derivatives, polyethylene glycols, silicones, bentonites, silicic acid, talc and zinc oxide or mixtures thereof.

Powders and sprays can contain, in addition to an active compound, excipients such as lactose, talc, silicic acid, aluminum hydroxide, calcium silicates and polyamide powder or mixtures of these substances. Sprays can additionally contain customary propellants, such as chlorofluorohydrocarbons and volatile unsubstituted hydrocarbons, such as butane and propane.

Transdermal patches have the added advantage of providing controlled delivery of a compound of the present invention to the body. Such dosage forms can be made by dissolving or dispersing the active compound in the proper medium. Absorption enhancers can also be used to increase the flux of the compound across the skin. The rate of such flux can be controlled by either providing a rate controlling membrane or dispersing the compound in a polymer matrix or gel.

Ophthalmic formulations, eye ointments, powders, solutions, and the like, are also contemplated as being within the scope of this invention. If desired, liquid ophthalmic formulations have properties similar to that of lacrimal fluids, aqueous humor or vitreous humor or are compatable with such fluids. A preferred route of administration is local administration (e.g., topical administration, such as eye drops or administration via an implant).

The phrases “parenteral administration” and “administered parenterally” as used herein means modes of administration other than enteral and topical administration, usually by injection and includes, without limitation, intravenous, intramuscular, intraarterial, intrathecal, intracapsular, intraorbital, intracardiac, intradermal, intraperitoneal, transtracheal, subcutaneous, subcuticular, intraarticular, subcapsular, subarachnoid, intraspinal and intrasternal injection and infusion.

Pharmaceutical compositions suitable for parenteral administration comprise one or more active compounds in combination with one or more pharmaceutically acceptable sterile isotonic aqueous or nonaqueous solutions, dispersions, suspensions or emulsions or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and nonaqueous carriers that may be employed in the pharmaceutical compositions of the invention include water, ethanol, polyols (such as glycerol, propylene glycol, polyethylene glycol and the like) and suitable mixtures thereof, vegetable oils, such as olive oil and injectable organic esters, such as ethyl oleate. Proper fluidity can be maintained, for example, by the use of coating materials, such as lecithin, by the maintenance of the required particle size in the case of dispersions and by the use of surfactants.

These compositions may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid and the like. It may also be desirable to include isotonic agents, such as sugars, sodium chloride and the like into the compositions. In addition, prolonged absorption of the injectable pharmaceutical form may be brought about by the inclusion of agents that delay absorption such as aluminium monostearate and gelatin.

In some cases, in order to prolong the effect of a drug, it is desirable to slow the absorption of the drug from subcutaneous or intramuscular injection. This may be accomplished by the use of a liquid suspension of crystalline or amorphous material having poor water solubility. The rate of absorption of the drug then depends upon its rate of dissolution, which, in turn, may depend upon crystal size and crystalline form. Alternatively, delayed absorption of a parenterally administered drug form is accomplished by dissolving or suspending the drug in an oil vehicle.

Injectable depot forms are made by forming microencapsulated matrices of the subject compounds in biodegradable polymers such as polylactide-polyglycolide. Depending on the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions that are compatible with body tissue.

For use in the methods of this invention, active compounds can be given per se or as a pharmaceutical composition containing, for example, 0.1 to 99.5% (more preferably, 0.5 to 90%) of active ingredient in combination with a pharmaceutically acceptable carrier.

Methods of introduction may also be provided by rechargeable or biodegradable devices. Various slow-release polymeric devices have been developed and tested in vivo in recent years for the controlled delivery of drugs, including proteinaceous biopharmaceuticals. A variety of biocompatible polymers (including hydrogels), including both biodegradable and non-degradable polymers, can be used to form an implant for the sustained release of a compound at a particular target site.

Actual dosage levels of the active ingredients in the pharmaceutical compositions may be varied so as to obtain an amount of the active ingredient that is effective to achieve the desired therapeutic response for a particular patient, composition, and mode of administration, without being toxic to the patient.

The selected dosage level will depend upon a variety of factors including the activity of the particular compound or combination of compounds employed or the ester, salt or amide thereof, the route of administration, the time of administration, the rate of excretion of the particular compound(s) being employed, the duration of the treatment, other drugs, compounds and/or materials used in combination with the particular compound(s) employed, the age, sex, weight, condition, general health and prior medical history of the patient being treated and like factors well known in the medical arts.

A physician or veterinarian having ordinary skill in the art can readily determine and prescribe the therapeutically effective amount of the pharmaceutical composition required. For example, the physician or veterinarian could start doses of the pharmaceutical composition or compound at levels lower than that required in order to achieve the desired therapeutic effect and gradually increase the dosage until the desired effect is achieved. By “therapeutically effective amount” is meant the concentration of a compound that is sufficient to elicit the desired therapeutic effect. It is generally understood that the effective amount of the compound will vary according to the weight, sex, age, and medical history of the subject. Other factors which influence the effective amount may include, but are not limited to, the severity of the patient's condition, the disorder being treated, the stability of the compound and, if desired, another type of therapeutic agent being administered with the compound of the invention. A larger total dose can be delivered by multiple administrations of the agent. Methods to determine efficacy and dosage are known to those skilled in the art (Isselbacher et al. (1996) Harrison's Principles of Internal Medicine 13 ed., 1814-1882, herein incorporated by reference).

In general, a suitable daily dose of an active compound used in the compositions and methods of the invention will be that amount of the compound that is the lowest dose effective to produce a therapeutic effect. Such an effective dose will generally depend upon the factors described above.

If desired, the effective daily dose of the active compound may be administered as one, two, three, four, five, six or more sub-doses administered separately at appropriate intervals throughout the day, optionally, in unit dosage forms. In yet another embodiment of the present invention, the active compound may be administered two or three times daily. In preferred embodiments, the active compound will be administered once daily.

The patient receiving this treatment is any animal in need, including primates, in particular humans and other mammals such as equines, cattle, swine and sheep; and poultry and pets in general.

Wetting agents, emulsifiers, and lubricants, such as sodium lauryl sulfate and magnesium stearate, as well as coloring agents, coating agents, release agents, sweetening, flavoring and perfuming agents, preservatives and antioxidants can also be present in the compositions.

Examples of pharmaceutically acceptable antioxidants include: (1) water-soluble antioxidants, such as ascorbic acid, cysteine hydrochloride, sodium bisulfate, sodium metabisulfite, sodium sulfite and the like; (2) oil-soluble antioxidants, such as ascorbyl palmitate, butylated hydroxyanisole (BHA), butylated hydroxytoluene (BHT), lecithin, propyl gallate, alpha-tocopherol and the like; and (3) metal-chelating agents, such as citric acid, ethylenediamine tetraacetic acid (EDTA), sorbitol, tartaric acid, phosphoric acid and the like.

In certain embodiments, the compounds of the present invention can be formulated and administered in a prodrug form. A prodrug is generally defined as a biologically inactive derivative of a parent drug molecule, which is activated by a chemical or enzymatic transformation within the body, which, in the process, involves the release of the active drug. In general, prodrugs comprise functional derivatives of the claimed compounds, which are capable of being enzymatically activated or converted into the more active parent form. Thus, in the treatment methods of the present invention, the term “administering” encompasses the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in Wihnan, 14 Biochem. Soc. Trans. 375-82 (1986); Stella et al., Prodrugs: A Chemical Approach to Targeted Drug Delivery in Directed Drug Delivery 247-67 (1985).

In certain embodiments, the present disclosure provides a compound of Formula (I) as described herein for use in preventing or treating a condition or a disease or a disorder caused by S. aureus in a subject. In certain embodiments, S. aureus is multidrug-resistant Staphylococcus aureus (MRSA) such as methicillin-resistant Staphylococcus aureus (MRSA), oxacillin-resistant Staphylococcus aureus (ORSA), gentamicin-resistant Staphylococcus aureus (GRSA), and azithromycin-resistant Staphylococcus aureus (ARSA).

In certain embodiments, the present disclosure provides a compound of Formula (I) for use in inhibiting S. aureus or other Gram-positive bacteria; wherein the compound of formula (1) is as described herein. In some embodiments, the present disclosure provides a compound of Formula (I) for use in inhibiting S. aureus; wherein the compound of formula (1) is as described herein. In certain embodiments, S. aureus is multidrug-resistant Staphylococcus aureus (MRSA) such as methicillin-resistant Staphylococcus aureus (MRSA), oxacillin-resistant Staphylococcus aureus (ORSA), gentamicin-resistant Staphylococcus aureus (GRSA), and azithromycin-resistant Staphylococcus aureus (ARSA).

The present disclosure further provides use of a compound of Formula (I) in the manufacture of a medicament. In some instances, the disclosure provides use of a compound of Formula (I), or a pharmaceutically acceptable salt, a pharmaceutically acceptable stereoisomer, a pharmaceutically acceptable hydrate, a pharmaceutically acceptable solvate, a prodrug, a polymorph, an N-oxide or a bioisostere thereof, in the manufacture of a medicament for preventing or treating a subject of a condition, disease or disorder where there is an advantage in inhibiting S. aureus or other Gram-positive bacteria. In some embodiments, the disclosure provides use of a compound of Formula (I), or a pharmaceutically acceptable salt, a pharmaceutically acceptable stereoisomer, a pharmaceutically acceptable hydrate, a pharmaceutically acceptable solvate, a prodrug, a polymorph, an N-oxide or a bioisostere thereof, in the manufacture of a medicament for preventing or treating a subject of a condition, disease or disorder where there is an advantage in inhibiting S. aureus. In certain embodiments, S. aureus is multidrug-resistant Staphylococcus aureus (MRSA) such as methicillin-resistant Staphylococcus aureus (MRSA), oxacillin-resistant Staphylococcus aureus (ORSA), gentamicin-resistant Staphylococcus aureus (GRSA), and azithromycin-resistant Staphylococcus aureus (ARSA).

In certain embodiments, the present disclosure further provides a compound of Formula (I) for use as a medicament or therapeutic agent in the prevention or treatment of a condition, disease, or disorder where there is an advantage in inhibiting S. aureus or other Gram-positive bacteria. In some embodiments, the present disclosure provides a compound of Formula (I) for use as a medicament or therapeutic agent in the prevention or treatment of a condition, disease, or disorder where there is an advantage in inhibiting S. aureus. In certain embodiments, S. aureus is multidrug-resistant Staphylococcus aureus (MRSA) such as methicillin-resistant Staphylococcus aureus (MRSA), oxacillin-resistant Staphylococcus aureus (ORSA), gentamicin-resistant Staphylococcus aureus (GRSA), and azithromycin-resistant Staphylococcus aureus (ARSA).

In certain embodiments, the present disclosure provides a method of preventing or treating a condition or a disease or a disorder caused by Staphylococcus aureus (S. aureus) in a subject; wherein the method comprises administering to the subject a therapeutically effective amount of a compound of Formula (I) provided herein. In certain embodiments, the present disclosure provides a method of treating a condition, disease, or disorder in a subject, wherein inhibition of S. aureus provides a benefit. The method comprises administering a therapeutically effective amount of a compound of Formula (I), or a pharmaceutically acceptable salt, a pharmaceutically acceptable stereoisomer, a pharmaceutically acceptable hydrate, a pharmaceutically acceptable solvate, a prodrug, a polymorph, an N-oxide or a bioisostere thereof, to a subject in need thereof. In certain embodiments, S. aureus is multidrug-resistant Staphylococcus aureus (MRSA) such as methicillin-resistant Staphylococcus aureus (MRSA), oxacillin-resistant Staphylococcus aureus (ORSA), gentamicin-resistant Staphylococcus aureus (GRSA), and azithromycin-resistant Staphylococcus aureus (ARSA).

In certain embodiments, the present invention provides a method of inhibiting S. aureus; wherein the method comprises administering to a subject in a need thereof a therapeutically effective amount of a compound of Formula (I) described herein. In certain embodiments, S. aureus is multidrug-resistant Staphylococcus aureus (MRSA) such as methicillin-resistant Staphylococcus aureus (MRSA), oxacillin-resistant Staphylococcus aureus (ORSA), gentamicin-resistant Staphylococcus aureus (GRSA), and azithromycin-resistant Staphylococcus aureus (ARSA).

The compounds of the present invention may be used as single drugs (monotherapy) or conjointly with one or more other agents (conjoint therapy). The compounds may be used by themselves or, preferably, in a pharmaceutical composition in which the compound is mixed with one or more pharmaceutically acceptable materials. The dosage of the compounds of the present invention varies depending on a patient's age, weight, or symptoms, as well as the compound's potency or therapeutic efficacy, the dosing regimen and/or treatment time. Generally, suitable routes of administration may, for example, include oral, eyedrop, rectal, transmucosal, topical or intestinal administration; parenteral delivery, including intramuscular, subcutaneous, intramedullary injections, as well as intrathecal, direct intraventricular, intravenous, intraperitoneal, intranasal, or intraocular injections. The compounds of the invention may be administered in an amount of 0.5 mg or 1 mg up to 500 mg, 1 g, or 2 g per dosage regimen. The dosage may be administered once per week, once per three days, once per two days, once per day, twice per day, three times per day or more often. In alternative embodiments, in certain adults the compound can be continuously administered by intravenous administration for a period of time designated by a physician. Since the dosage is affected by various conditions, an amount less than or greater than the dosage ranges contemplated about may be implemented in certain cases. A physician can readily determine the appropriate dosage for a patient undergoing therapeutic treatment.

The compounds of the present invention may be administered in combination with one or more other drugs (1) to complement and/or enhance effect of the compound of the present invention, (2) to modulate pharmacodynamics, improve absorption or reduce dosage of the compound of the present invention and/or (3) to reduce or ameliorate the side effects of the compound of the present invention. As used herein, the phrase “conjoint administration” refers to any form of administration of two or more different therapeutic compounds such that the second compound is administered while the previously administered therapeutic compound is still effective in the body (e.g., the two compounds are simultaneously effective in the patient, which may include synergistic effects of the two compounds). For example, the different therapeutic compounds can be administered either in the same formulation or in a 24-separate formulation, either concomitantly or sequentially. In yet another embodiment, the different therapeutic compounds can be administered within one hour, 12 h, 24 h, 36 h, 48 h, 72 h or a week of one another. Thus, an individual who receives such treatment can benefit from a combined effect of different therapeutic compounds. The respective compounds may be administered by the same or different route and the same or different method.

The dosage of the other drug can be a dosage that has been clinically used or may be a reduced dosage that is effective when administered in combination with a compound of the present invention. The ratio of the compound of the present invention and the other drug can vary according to age and weight of a subject to be administered, administration method, administration time, disorder to be treated, symptom and combination thereof. For example, the other drug may be used in an amount of 0.01 to 100 parts by mass, based on 1 part by mass of the compound of the present invention.

The compounds of present disclosure may be formulated and administered in a prodrug form. In general, prodrugs comprise functional derivatives of the claimed compounds which are capable of being enzymatically activated or converted into the more active parent form. Thus, in the treatment methods of the present invention, the term “administering” encompasses the treatment of the various disorders described with the compound specifically disclosed or with a compound which may not be specifically disclosed, but which converts to the specified compound in vivo after administration to the patient. Conventional procedures for the selection and preparation of suitable prodrug derivatives are described, for example, in Wihnan, 14 Biochem. Soc. Trans. 375-82 (1986); Stella et al., Prodrugs: A Chemical Approach to Targeted Drug Delivery in Directed Drug Delivery 247-67 (1985).

In certain embodiments, different compounds of the invention may be conjointly administered with one or more other compounds of the invention. Moreover, such combinations may be conjointly administered with other therapeutic agents, such as other agents suitable for the treatment of diseases and/or disorders described herein.

The present invention also provides a process for the preparation of the compounds of Formula (I) are set forth in the below Examples and generalized Scheme. One of skill in the art will recognize that scheme can be adapted to produce the compounds of general Formula (I) and pharmaceutically acceptable salts or stereo isomers of compounds of general Formula (I) according to the present invention.

The schemes are given for the purpose of illustrating the invention and are not intended to limit the scope or spirit of the invention. Starting materials shown in the schemes can be obtained from commercial sources or prepared based on procedures described in the literature. Furthermore, in the following schemes, where specific acids, bases, reagents, coupling agents, solvents, etc. are mentioned, it is understood that other suitable acids, bases, reagents, coupling agents etc. may be used and are included within the scope of the present invention. Modifications to reaction conditions, for example, temperature, duration of the reaction or combinations thereof, are envisioned as part of the present invention. All possible stereoisomers are envisioned within the scope of this invention.

The intermediates required for the synthesis are commercially available or alternatively, these intermediates can be prepared using known literature methods. The invention is described in greater detail by way of specific examples.

It is contemplated that some of the intermediates disclosed in the present invention are used for the next step without any characterization data.

It is meant to be understood that the order of the steps in the processes may be varied, that reagents, solvents, and reaction conditions may be substituted for those specifically mentioned, and that vulnerable moieties may be protected and deprotected, as necessary. The variables “R”, “Ar” and “Het” independently represent all the possible substitutions as disclosed in compound of Formula (I).

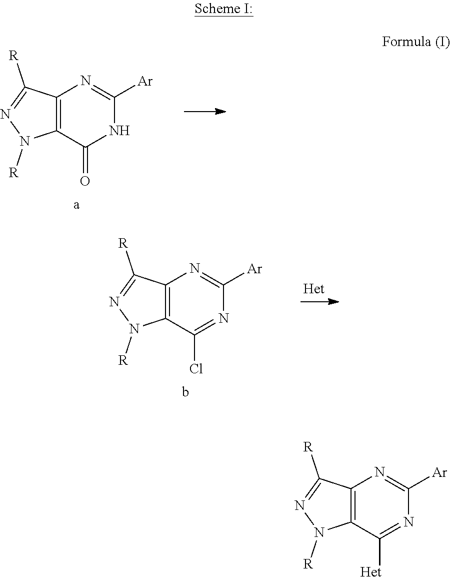

The compounds of the present invention are prepared by reacting compound of formula a (prepared as shown in Example 1 step iii of WO2006058201 which is herein incorporated by reference) with phosphorous chloride in the absence or presence of a suitable solvent selected from benzene, toluene, xylene, THF, 1,4-dioxane, diethyl ether, diphenyl ether, dichloromethane, chloroform, 1,2-dichloroethane and the likes thereof to give compound of formula b which is then reacted with suitable ‘Het’ compound in presence of aluminium chloride and suitable solvent selected from nitrobenzene, THF, 1,4-dioxane, diethyl ether, diphenyl ether, dichloromethane, chloroform, 1,2-dichloroethane or the likes thereof to give a compound of Formula (I).

It is understood that in any of the above schemes, any reactive group in the substrate molecule may be protected according to any conventional procedure known in the prior art. Suitable protecting groups comprise N-Boc, N-Cbz, N-Fmoc, alkyl, benzophenoneimine for protection of amino group, acetal protection for aldehyde, ketal protection for ketone. The invention also encompasses prodrugs of compounds of the invention, which on administration undergo chemical conversion by metabolic processes before becoming active pharmacological substances. In general, such prodrugs will be functional derivatives of compounds of invention, which are readily convertible in vivo into compounds of the invention. The invention also encompasses the active metabolites of the compounds of the present invention of general Formula (I).

### EXAMPLES

The following examples are given by way of illustration of the working of the invention in actual practice and therefore should not be construed to limit the scope of present invention.

### Example 1: General Method of Synthesis of Compounds of the Invention

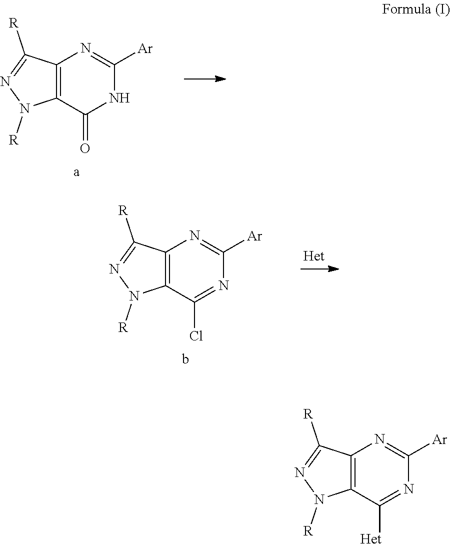

Unless stated otherwise, reactions were performed under nitrogen atmosphere. Reactions were monitored by thin layer chromatography (TLC) on silica gel plates (60 F254), visualizing with ultraviolet light or iodine spray. Flash chromatography was performed on silica gel (100-200 mesh) using distilled hexene, ethyl acetate, dichloromethane. ¹H NMR and ¹³C NMR spectra were recorded either in CDCl₃, DMSO-d₆ or CF₃CO₂H solution by using 400 and 100 MHz spectrometers, respectively. Proton chemical shifts (δ) are relative to tetramethylsilane (TMS, δ 0.00) as internal standard and expressed in ppm. Spin multiplicities are given as s (singlet), d (doublet), t (triplet) and m (multiplet) as well as b (broad). Coupling constants (J) are given in hertz. Infrared spectra were recorded on a FT-IR spectrometer. Melting points were determined using melting point apparatus and are uncorrected. MS spectra were obtained on a mass spectrometer. Mass spectra (MS) and high-resolution mass spectra (HRMS) were recorded using electron ionization (EI) mass spectrometry.

Preparation of compound b: A mixture of compound of formula a (4 g, 13.24 mmol) and phosphorous oxychloride (40 mL) was stirred at 100° C., for 12-14 h under anhydrous conditions. After completion of the reaction excess of phosphorous oxychloride was removed under vacuum. The residue was diluted with aq NaHCO₃ solution to reach the pH=7-8. White solid separated was filtered, washed with water (2×30 mL) and dried under vacuum to afford compound of formula b.

Preparation of compound of Formula (I): A mixture of compound of formula b, an appropriate ‘Het’ group and AlCl₃ (1.2 equiv) in dichloroethane (5 mL) was stirred at 80° C. for 7-12 h under a nitrogen atmosphere. After completion of the reaction, the mixture was poured into ice-cold water (15 mL), stirred for 10 min, and then extracted with ethyl acetate (3×20 mL). The organic layers were collected, combined, washed with cold water (2×20 mL), dried over anhydrous Na₂SO₄ and concentrated under vacuum. The residue obtained was purified by column chromatography using ethyl acetate/hexene to afford the compound of Formula (I). Following are the synthesis of specific compounds of the invention.

### Compound 1: Preparation of 5-(4-chlorophenyl)-7-(1H-indol-3-yl)-1-methyl-3-propyl-1H-pyrazolo[4,3-d]pyrimidine

The compound was prepared by reacting compound of formula b (wherein one of the R groups is propyl and the other R group is methyl and Ar is chloro substituted aryl group) with indole following the procedure given above in the preparation of compound of Formula (I).

Yield: 75%; White solid; mp 110-112° C.; ¹H NMR (400 MHz, DMSO-d₆) δ 11.96 (bs, 1H), 8.50 (d, J=8.8 Hz, 2H), 8.19-8.15 (m, 2H), 7.61-7.56 (m, 3H), 7.29-7.21 (m, 2H), 4.03 (s, 3H), 3.02 (t, J=7.4 Hz, 2H), 1.98-1.88 (m, 2H), 1.02 (t, J=7.6 Hz, 3H)¹³C NMR (100 MHz, DMSO-d₆) δ 148.3, 145.0, 144.5, 137.2 (2C), 136.6, 134.5, 130.4, 129.2 (2C), 128.7 (2C), 126.4, 122.6 (2C), 120.9, 120.5, 112.2, 111.2, 38.2, 27.3, 21.6, 14.0; HPLC: 91.74%, column: X Bridge C-18 150*4.6 mm 5μ, mobile phase A: 0.05% Formic Acid in water mobile phase B: CH₃CN, gradient (T/% B): 0/90, 10/95, 12/95, 15/90, 18/90; flow rate: 1.0 mL/min; UV 210 nm, retention time 5.67 min; IR (KBr) vₘₐₓ 3277, 2957, 1918, 1538, 1437 cm⁻¹; Mass (ESI method): 402.2 (M+1, 100%), HRMS (ESI) calcd for C₂₃H₂₁N₅Cl (M+H)⁺ 402.1485, found 402.1497.

### Compound 2: Preparation of 5-(4-chlorophenyl)-1-methyl-7-(1-methyl-1H-indol-3-yl)-3-propyl-1H-pyrazolo[4,3 d]pyrimidine

The compound was prepared by reacting compound of formula b (wherein one of the R groups is propyl and the other R group is methyl and Ar is chloro substituted aryl group) with 1-methyl-indole following the procedure given above in the preparation of compound of Formula (I).

Yield: 75%; White solid; mp: 180-181° C.; ¹H NMR (400 MHz, CDCl₃) δ 8.57 (d, J=8.4 Hz, 2H), 8.03 (d, J=7.6 Hz, 1H), 7.59 (s, 1H), 7.46-7.43 (m, 3H), 7.38 (t, J=7.4 Hz, 1H) 7.31-7.28 (m, 1H), 3.96 (s, 6H), 3.14-3.10 (m, 2H), 2.04-1.97 (m, 2H), 1.10 (t, J=7.4 Hz, 3H); ¹³C NMR (100 MHz, CDCl₃) δ 156.2, 147.2, 147.0, 145.5, 137.3, 137.2, 135.5, 131.3, 129.8, 129.4 (2C), 128.5 (2C), 127.2, 123.1, 121.4, 121.0, 112.1, 109.8, 38.9, 33.4, 27.9, 22.1, 14.1; HPLC: 98.59%, column: X Bridge C-18 150*4.6 mm 5μ, mobile phase A: 0.05% Formic Acid in water mobile phase B: CH₃CN, gradient (T/% B): 0/90, 2/90, 8/98, 10/98, 10/98, 12/90, 15/90; flow rate: 1.0 mL/min; UV 265 nm, retention time 5.74 min; IR (KBr) vₘₐₓ 2954, 1916, 1539, 1232 cm⁻¹; HRMS (ESI) calcd for C₂₄H₂₃N₅Cl (M+H)⁺ 416.1642, found 416.1653.

### Compound 3: Preparation of 5-(4-chlorophenyl)-7-(1-ethyl-1H-indol-3-yl)-1-methyl-3-propyl-1H-pyrazolo[4,3-d]pyrimidine

The compound was prepared by reacting compound of formula b (wherein one of the R groups is propyl and the other R group is methyl and Ar is chloro substituted aryl group) with 1-ethyl-indole following the procedure given above in the preparation of compound of Formula (I).

Yield: 75%; White solid; mp 160-162° C.; ¹H NMR (400 MHz, CDCl₃) δ 8.55 (d, J=8.8 Hz, 2H), 8.06 (d, J=8.4 Hz, 1H), 7.65 (s, 1H), 7.49-7.44 (m, 3H), 7.36-7.28 (m, 2H), 4.33 (q, J=7.2 Hz, 2H), 3.96 (s, 3H), 3.12 (t, J=7.4 Hz, 2H), 2.04-1.95 (m, 2H), 1.60 (t, J=8.0 Hz, 3H), 1.10 (t, J=7.2 Hz, 3H); ¹³C NMR (100 MHz, CDCl₃) δ 151.1, 142.3, 141.9, 140.4, 132.1, 131.2, 130.3, 124.7, 124.6 (2C), 124.3, 123.4, 123.3, 122.3, 117.9, 116.2, 116.0, 107.0, 104.8, 36.4, 33.8, 22.8, 17.0, 10.3, 9.0; HPLC: 99.29%, column: X Bridge C-18 150*4.6 mm 5μ, mobile phase A: 0.05% Formic Acid in water mobile phase B: CH₃CN, gradient (T/% B): 0/90, 2/90, 8/98, 10/98, 12/90, 15/90; flow rate: 1.0 mL/min; UV 265 nm, retention time 6.41 min; IR (KBr) vₘₐₓ 2978, 2871, 1912, 1543, 1214 cm⁻¹; HRMS (ESI) calcd for C₂₅H₂₅N₅Cl (M+H)⁺ 430.1798, found 430.1815.

### Compound 4: Preparation of 7-(5-bromo-1H-indol-3-yl)-5-(4-chlorophenyl)-1-methyl-3-propyl-1H-pyrazolo[4,3-d]pyrimidine

The compound was prepared by reacting compound of formula b (wherein one of the R groups is propyl and the other R group is methyl and Ar is chloro substituted aryl group) with 5-bromo-indole following the procedure given above in the preparation of compound of Formula (I).

Yield: 73%; White solid; mp 196-198° C.; ¹H NMR (400 MHz, CDCl₃) δ 8.72 (bs, 1H), 8.54 (m, 2H), 8.21 (d, J=1.2 Hz, 1H), 7.69 (d, J=2.8 Hz, 1H), 7.48-7.38 (m, 4H), 3.95 (s, 3H), 3.12 (t, J=7.6 Hz, 2H), 2.04-1.95 (m, 2H), 1.10 (t, J=7.2 Hz, 3H); ¹³C NMR (100 MHz, CDCl₃) δ 156.2, 147.1, 146.5, 145.7, 137.0, 135.7, 134.8, 129.5, 129.3 (2C), 128.6 (2C), 128.3, 127.8, 126.5, 123.5, 114.9, 113.1, 112.5, 38.9, 27.9, 22.0, 14.1; HPLC: 92.55%, column: X Bridge C-18 150*4.6 mm 5μ, mobile phase A: 0.05% Formic Acid in water mobile phase B: CH₃CN, gradient (T/% B): 0/90, 10/95, 12/95, 15/90, 18/90; flow rate: 1.0 mL/min; UV 210 nm, retention time 8.50 min; IR (KBr) vₘₐₓ 3419, 3155, 2955, 1897, 1543 cm⁻¹; HRMS (ESI) calcd for C₂₃H₂₀N₅ClBr (M+H)⁺ 480.0591, found 480.0591.

### Compound 5: Preparation of 7-(5-bromo-1-methyl-1H-indol-3-yl)-5-(4-chlorophenyl)-1-methyl-3-propyl-1H-pyrazolo[4,3-d]pyrimidine

The compound was prepared by reacting compound of formula b (wherein one of the R groups is propyl and the other R group is methyl and Ar is chloro substituted aryl group) with 5-bromo-1-methyl-indole following the procedure given above in the preparation of compound of Formula (I).

Yield: 72%; White solid; mp 237-238° C.; ¹H NMR (400 MHz, DMSO-d₆) δ 8.48 (d, J=8.4 Hz, 2H), 8.33 (s, 1H), 8.27 (s, 1H), 7.65-7.48 (m, 3H), 7.47-7.46 (m, 1H), 4.06 (s, 3H), 3.97 (s, 3H), 3.04-3.01 (m, 2H), 1.93-1.88 (m, 2H), 1.02 (t, J=7.4 Hz, 3H); ¹³C NMR (100 MHz, TFA-d) δ 152.5, 151.7, 144.9, 141.3, 139.7, 137.2, 135.1, 129.7, 129.5, 128.7, 128.4, 127.9, 127.5, 123.8, 118.8, 118.2, 115.5, 112.7, 112.3, 38.7, 33.0, 26.4, 21.7, 11.8; HPLC: 99.91%, column: X Bridge C-18 150*4.6 mm 5μ, mobile phase A: 0.05% Formic Acid in water mobile phase B: CH₃CN, gradient (T/% B): 0/90, 2/90, 8/98, 10/98, 12/90, 15/90; flow rate: 1.0 mL/min; UV 265 nm, retention time 7.95 min; IR (KBr) vₘₐₓ 3133, 2970, 1929, 1582, 1433 cm⁻¹; HRMS (ESI) calcd for C₂₄H₂₂N₅ClBr (M+H)⁺ 494.0747, found 494.0763.

### Compound 6: Preparation of 7-(5-bromo-1-ethyl-1H-indol-3-yl)-5-(4-chlorophenyl)-1-methyl-3-propyl-1H-pyrazolo[4,3-d]pyrimidine

The above compound was prepared by reacting compound of formula b (wherein one of the R groups is propyl and the other R group is methyl and Ar is chloro substituted aryl group) with 1-ethyl-5-bromo-indole following the procedure given above in the preparation of compound of Formula (I).

Yield: 75%; White solid; mp 212-213° C.; ¹H NMR (400 MHz, TFA-d) δ 8.54 (s, 1H), 8.38 (s, 1H), 8.28-8.26 (m, 2H), 7.74-7.71 (m, 3H), 7.60-7.58 (m, 1H), 4.54 (q, J=4.0, 2H), 4.40 (s, 3H), 3.28 (t, J=7.2 Hz, 2H), 2.02 (q, J=7.2 Hz, 2H), 1.73 (t, J=7.2 Hz, 3H), 1.17 (t, J=6.8 Hz, 3H); ¹³C NMR (100 MHz, TFA-d) δ 152.6, 151.9, 144.9, 141.4, 138.3, 136.3, 134.9, 129.7, 129.6, 128.6, 128.5, 128.0, 127.8, 124.0, 118.4, 118.2, 115.6, 112.8, 112.5, 42.7, 38.7, 26.5, 21.7, 13.2, 11.8; HPLC: 99.86%, column: X Bridge C-18 150*4.6 mm 5μ, mobile phase A: 0.05% Formic Acid in water mobile phase B: CH₃CN, gradient (T/% B): 0/90, 2/90, 8/98, 10/98, 12/90, 15/90; flow rate: 1.0 mL/min; UV 230 nm, retention time 8.80 min; IR (KBr) vₘₐₓ 2956, 2870, 1737, 1582, 1219 cm⁻¹; HRMS (ESI) calcd for C₂₅H₂₄N₅ClBr (M+H)⁺ 508.0904, found 508.0910.

### Compound 7: Preparation of 7-(5-chloro-1H-indol-3-yl)-5-(4-chlorophenyl)-1-methyl-3-propyl-1H-pyrazolo[4,3-d]pyrimidine

The compound was prepared by reacting compound of formula b (wherein one of the R groups is propyl and the other R group is methyl and Ar is chloro substituted aryl group) with 5-chloro-indole following the procedure given above in the preparation of compound of Formula (I).

Yield: 76%; White solid; mp 230-232° C.; ¹H NMR (400 MHz, CDCl₃) δ 8.79 (bs, 1H), 8.54 (d, J=9.2 Hz, 2H), 8.04 (d, J=2.0 Hz, 1H), 7.68 (d, J=3.2 Hz, 1H), 7.47-7.40 (m, 3H), 7.31-7.25 (m, 1H), 3.94 (s, 3H), 3.12 (t, J=7.6 Hz, 2H), 2.04-1.97 (m, 2H), 1.10 (t, J=7.4 Hz, 3H); ¹³C NMR (100 MHz, CDCl₃) δ 156.2, 147.1, 146.5, 145.7, 136.9, 135.7, 134.5 (2C), 129.3 (2C), 128.6 (2C), 127.9, 127.6, 127.5, 124.0, 120.3, 113.2, 112.6, 38.9, 29.6, 22.1, 14.1; HPLC: 96.30%, column: X Bridge C-18 150*4.6 mm 5μ, mobile phase A: 0.05% Formic Acid in water mobile phase B: CH₃CN, gradient (T/% B): 0/90, 2/90, 8/98, 10/98, 12/90, 15/90; flow rate: 1.0 mL/min; UV 265 nm, retention time 6.15 min; IR (KBr) vₘₐₓ 3206, 3170, 2957, 1541, 1109 cm⁻¹; HRMS (ESI) calcd for C₂₃H₂₀N₅Cl₂(M+H)+436.1109, found 436.1096.

### Compound 8: Preparation of 7-(5-chloro-6-fluoro-1H-indol-3-yl)-5-(4-chlorophenyl)-1-methyl-3-propyl-1H-pyrazolo[4,3-d]pyrimidine

The compound was prepared by reacting compound of formula b (wherein one of the R groups is propyl and the other R group is methyl and Ar is chloro substituted aryl group) with 5-chloro-6-fluoro-indole following the procedure given above in the preparation of compound of Formula (I).

Yield: 80%; White solid; mp 231-232° C.; ¹H NMR (400 MHz, CDCl₃) δ 8.80 (bs, 1H), 8.52 (d, J=8.4 Hz, 2H), 7.85 (d, J=9.6 Hz, 1H), 7.70 (d, J=2.4 Hz, 1H), 7.53 (d, J=5.6 Hz, 1H) 7.45 (d, J=8.4 Hz, 2H), 3.97 (s, 3H) 3.12 (t, J=7.6 Hz, 2H), 2.02-1.96 (m, 2H), 1.08 (t, J=7.2 Hz, 3H); ¹³C NMR (100 MHz, CDCl₃) δ 156.2, 155.4, 153.0, 147.2, 146.2, 145.9, 136.9, 135.8, 132.4 (2C), 129.3 (2C), 128.6 (2C), 128.5, 125.6, 112.8, 107.3, 107.1, 38.96, 27.8, 22.0, 14.1; HPLC: 99.83%, column: X Bridge C-18 150*4.6 mm 5μ, mobile phase A: 0.05% Formic Acid in water mobile phase B: CH₃CN, gradient (T/% B): 0/90, 2/90, 8/98, 10/98, 12/90, 15/90; flow rate: 1.0 mL/min; UV 265 nm, retention time 6.0 min; IR (KBr) vₘₐₓ 3169, 2953, 2869, 1728, 1462 cm⁻¹; HRMS (ESI) calcd for C₂₃H₁₉N₅Cl₂F (M+H)⁺ 454.1002, found 454.0999.

### Compound 9: 5-(4-chlorophenyl)-7-(5,6-difluoro-1H-indol-3-yl)-1-methyl-3-propyl-1H-pyrazolo[4,3-d]pyrimidine

The compound was prepared by reacting compound of formula b (wherein one of the R groups is propyl and the other R group is methyl and Ar is chloro substituted aryl group) with 5,6-difluoro-indole following the procedure given above in the preparation of compound of Formula (I).

Yield: 78%; Brown solid; mp 233-234° C.; ¹H NMR (400 MHz, CDCl₃) δ 8.69 (bs, 1H), 8.52 (m, 2H), 7.91-7.86 (m, 1H), 7.69 (s, 1H), 7.47 (d, J=8.8 Hz, 2H) 7.32-7.27 (m, 1H), 3.99 (s, 3H) 3.12 (t, J=7.6 Hz, 2H), 2.04-1.94 (m, 2H), 1.09 (t, J=7.4 Hz, 3H). ¹³C NMR (100 MHz, CDCl₃) δ 156.2, 155.4, 153.0, 147.2, 146.3, 145.9, 136.9 (2C), 135.7, 129.3 (2C), 128.6 (2C), 127.8, 125.6, 108.0, 107.8, 99.7, 99.5, 38.9, 27.8, 22.1, 14.1; HPLC: 98.59%, column: X Bridge C-18 150*4.6 mm 5μ, mobile phase A: 0.05% Formic Acid in water mobile phase B: CH₃CN, gradient (T/% B): 0/90, 2/90, 8/98, 10/98, 12/90, 15/90; flow rate: 1.0 mL/min; UV 265 nm, retention time 5.22 min; IR (KBr) vₘₐₓ 3131, 2959, 1920, 1584, 1175 cm⁻¹ HRMS (ESI) calcd for C₂₃H₁₉N₅ClF₂ (M+H)⁺ 438.1297, found 438.1297.

### Compound 10: 5-(4-chlorophenyl)-7-(5-methoxy-1H-indol-3-yl)-1-methyl-3-propyl-1H-pyrazolo[4,3-d]pyrimidine

The compound was prepared by reacting compound of formula b (wherein one of the R groups is propyl and the other R group is methyl and Ar is chloro substituted aryl group) with 5-methoxy-indole following the procedure given above in the preparation of compound of Formula (I).

Yield: 66%; white solid; mp: 183-185° C.; R_{f}=0.6 (20% EtOAc/n-hexane); ¹H NMR (400 MHz, CDCl₃) δ: 8.60 (dd, J=8.61, 6.23 Hz, 3H), 7.65 (dd, J=10.33, 2.52 Hz, 2H), 7.45 (d, J=8.66 Hz, 2H), 7.43-7.33 (m, 1H), 7.01 (dd, J=8.85, 2.44 Hz, 1H), 4.01 (s, 3H), 3.85 (s, 3H), 3.26-2.96 (t, J=8 Hz, 2H), 2.00 (dt, J=14.75, 7.18 Hz, 2H), 1.10 (t, J=7.36 Hz, 3H); ¹³CNMR (100 MHz, CDCl₃) δ 156.1, 155.6, 147.4, 147.0, 145.6, 135.6, 131.2, 129.6 (2C), 129.3, 128.5 (2C), 127.4, 127.2, 114.3, 113.3, 112.4 (2C), 102.2, 55.7, 39.0, 27.9, 22.1, 14.1; MS (ES mass): 431.2; HPLC: 97.66%, Column: X-Bridge C-18 150*4.6 mm, 5 μm, mobile phase A: 0.1% formic acid in water, mobile phase B: CH₃CN, (T/% B): 0/10, 3/20, 10/95, 20/95, 22/20, 25/20; flow rate: 1.0 mL/min; Diluent: water:CAN (80:20); UV 254.5 nm, retention time 13.6 min.

### Compound 11: 4-chlorophenyl)-7-(5-methoxy-1-methyl-1H-indol-3-yl)-1-methyl-3-propyl-1H-pyrazolo[4,3-d]pyrimidine

The compound was prepared by reacting compound of formula b (wherein one of the R groups is propyl and the other R group is methyl and Ar is chloro substituted aryl group) with 5-methoxy-1-methyl-indole following the procedure given above in the preparation of compound of Formula (I).

Yield: 71%; white solid; mp: 152-154° C.; R_{f}=0.5.1 (20% EtOAc/n-hexane); ¹H NMR (400 MHz, CD Cl₃) δ:8.40 (d, J=8.68 Hz, 2H), 7.45 (d, J=2.38 Hz, 1H), 7.37 (s, 1H), 7.27 (d, J=8.65 Hz, 2H), 7.17 (d, J=8.89 Hz, 1H), 6.86 (dd, J=8.93, 2.43 Hz, 1H), 3.85 (s, 3H), 3.76 (s, 3H), 3.68 (s, 3H), 2.90 (t, J=8 Hz, 2H), 1.96-1.70 (m, 2H), 1.06 (t, J=7.36 Hz, 3H); ¹³CNMR (100 MHz, CDCl₃) δ 156.0, 155.5, 147.0, 135.5, 132.5 (2C), 131.8, 129.3 (2C), 128.5 (2C), 127.7 (2C), 113.8, 111.5, 110.7, 109.9 (2C), 102.4, 55.7, 39.0, 33.6, 27.9, 22.1, 14.1; MS (ES mass): 445.2; HPLC: 95.63%, Column: X-Terra RP18 250*4.6 mm, 5 μm, mobile phase A: 0.1% TFA in water, mobile phase B: CH₃CN, (T/% B): 0/20, 5/20, 15/95, 23/95, 25/20, 30/20; flow rate: 1.0 mL/min; Diluent: water:CAN (80:20); UV 254.5 nm, retention time 19.7 min.

### Compound 12: 5-(4-chlorophenyl)-7-(1-ethyl-5-methoxy-1H-indol-3-yl)-1-methyl-3-propyl-1H-pyrazolo[4,3-d]pyrimidine

The compound was prepared by reacting compound of formula b (wherein one of the R groups is propyl and the other R group is methyl and Ar is chloro substituted aryl group) with 1-ethyl-5-methoxy-indole following the procedure given above in the preparation of compound of Formula (I).

Yield: 79%; white solid; mp: 147-149° C.; R_{f}=0.7 (20% EtOAc/n-hexane); ¹H NMR (400 MHz, CDCl₃) δ: 8.57 (t, J=14.15 Hz, 2H), 7.79-7.59 (m, 2H), 7.47 (dd, J=19.17, 8.69 Hz, 2H), 7.37 (d, J=8.93 Hz, 1H), 7.03 (dd, J=8.93, 2.46 Hz, 1H), 4.49-4.21 (m, 2H), 4.04 (s, 3H), 3.93-3.81 (m, 3H), 3.26-3.07 (t, J=8 Hz, 2H), 2.09-1.90 (m, 2H), 1.58 (t, J=7.29 Hz, 3H), 1.11 (t, J=7.36 Hz, 3H); ¹³C NMR (100 MHz, CDCl₃) δ: 156.1, 155.5, 147.5, 147.1, 145.6, 137.3, 135.5, 131.6, 130.1, 129.5, 129.3, 128.5, 127.8, 113.7, 111.5, 110.7, 102.5, 55.7, 41.7, 39.0, 31.4, 30.1, 27.9, 22.1, 15.5, 14.2; MS (ES mass); 460.0; HPLC: 99.55%, Column: X-Terra RP18 250*4.6 mm, 5 μm, mobile phase A: 0.1% TFA in water, mobile phase B: CH₃CN, (T/% B): 0/20, 5/20, 15/95, 23/95, 25/20, 30/20; flow rate: 1.0 mL/min; Diluent: water:ACN (80:20); UV 254.5 nm, retention time 16.52 min.

### Compound 13: 7-(1-allyl-1H-indol-3-yl)-5-(4-chlorophenyl)-1-methyl-3-propyl-1H-pyrazolo[4,3-d]pyrimidine

The compound was prepared by reacting compound of formula b (wherein one of the R groups is propyl and the other R group is methyl and Ar is chloro substituted aryl group) with 1-allyl-indole following the procedure given above in the preparation of compound of Formula (I).

Yield: 71%; white solid; mp: 137-139° C.; R_{f}=0.6; (20% EtOAc/n-hexane); ¹H NMR (400 MHz, CDCl₃) δ: 8.55 (dd, J=11.59, 8.76 Hz, 2H), 8.09 (d, J=7.85 Hz, 1H), 7.78 (s, 1H), 7.50-7.43 (m, 3H), 7.36 (m, 2H), 6.23-5.96 (m, 1H), 5.31 (dd, J=29.80, 13.69 Hz, 2H), 4.89 (d, J=5.55 Hz, 2H), 4.00 (s, 3H), 3.20 (t, J=7.62 Hz, 2H), 2.00 (m, 2H), 1.10 (t, J=7.36 Hz, 3H); ¹³C NMR (100 MHz, CDCl₃) δ:156.1, 147.3, 147.0, 145.5, 137.2, 136.7 (2C), 135.5, 132.5, 130.4, 129.7, 129.4, 128.5, 127.4, 123.2, 121.5, 121.2 (2C), 118.5, 112.4, 110.2, 49.2, 38.9, 27.9, 22.1, 14.2; MS (ES mass): 441.9; HPLC: 99.47%, Column: X-Terra RP18 250*4.6 mm, 5 μm, mobile phase A: 0.1% TFA in water, mobile phase B: CH₃CN, (T/% B): 0/20, 5/20, 15/95, 23/95, 25/20, 30/20; flow rate: 1.0 mL/min; Diluent: water:ACN (80:20); UV 254.5 nm, retention time 17.05 min.

### Example 2: Inhibitory Effect of Compound 7 on Different Strains of Staphylococcus aureus

Compound 7 was tested to evaluate its inhibitory effect on Staphylococcus aureus. An experiment was conducted to assess the minimum inhibitory concentration (MIC) of the compound of Formula (I).

### List of Chemicals, Reagents and Media Used in the Study:

DMSO, sterile saline, sterile ultrapure water, soyabean casein digest agar (SCDA), and Cation Adjusted Mueller Hinton Broth (CAMHB).

### Preparation of Test Compound

20.0 mg of test compound was weighed and dissolved in 0.5 mL of filtered DMSO (100%) to prepare a stock solution of 40 mg/mL concentration. Initial stock (Stock 1 and Stock 2) and working stock solution (10 concentrations) were prepared as mentioned in the table below. Final testing concentration in the micro-titer plates was starting from 1000 μg/mL in experiment plate.

| | Initial stock concentration | | | Working stock concentration | | | |
|---|---|---|---|---|---|---|---|
| | Preparation | | | preparation | | | |
| | | Quantity | | | Quantity | | Working |
| | | from | Quantity | | from | Quantity of | stock |
| S. | Stock 1 | Stock1 | of DMSO | Stock 2 | Stock 2 | CAMHB | concentration |
| No | (mg/mL) | (μL) | (μL) | (mg/mL) | (μL) | (μL) | (μg/mL) |
| 1 | 40.00 | NA | NA | 40.00 | 200 | 3800 | 2000.0 |
| 2 | 40.00 | NA | NA | 40.00 | 100 | 3900 | 1000.0 |
| 3 | 40.00 | NA | NA | 40.00 | 50 | 3950 | 500.0 |
| 4 | 40.00 | 100.00 | 100.00 | 20.00 | 50 | 3950 | 250.0 |
| 5 | 20.00 | 100.00 | 100.00 | 10.00 | 50 | 3950 | 125.0 |
| 6 | 10.00 | 100.00 | 100.00 | 5.00 | 50 | 3950 | 63.0 |
| 7 | 5.00 | 100.00 | 100.00 | 2.50 | 50 | 3950 | 31.0 |
| 8 | 2.50 | 100.00 | 100.00 | 1.25 | 50 | 3950 | 16.0 |
| 9 | 1.25 | 100.00 | 100.00 | 0.63 | 50 | 3950 | 8.0 |
| 10 | 0.63 | 100.00 | 100.00 | 0.31 | 50 | 3950 | 4.0 |

*The final concentration of the test compound will reduce by half on addition of culture. For Staphylococcus aureus 0.64 μg/mL was prepared, and 50 μL of stock was diluted in 3950 μL CAMHB to make 8.0 μg/mL and serially diluted upto ten dilutions (for plate 2).

### Preparation of Reference Standard

The reference standards gentamicin, norfloxacin and levofloxacin were dissolved using sterile ultrapure water to prepare a stock solution of 2.0 mg/mL concentration. Working stock solution of 64 μg/mL was prepared in CAMHB by diluting 0.032 mL of 2.0 mg/mL stock solution in 0.968 mL of growth medium. The testing concentration in the micro-titer plates is 32 μg/mL.

### List of Tester Strains and Growth Conditions

| | | | | Growth |
|---|---|---|---|---|
| | | | | medium |
| S. | | | Anthem | and |
| No. | Organism | Source | No. | conditions |
| 1 | Escherichia coli | ATCC9637 | AB1740 | CAMHB, |
| 2 | Pseudomonas | ATCC 9027 | AB201 | 37° C. |
| | aeruginosa | | | for 18-20 |
| 3 | Staphylococcus aureus | ATCC25923 | AB002 | hours |
| 4 | Staphylococcus aureus | ATCC700699 | AB139 | |
| 5 | Staphylococcus aureus | ATCC29213 | AB001 | |
| 6 | Staphylococcus aureus | ATCC43300 | AB137 | |
| | (Gentamicin resistant) | | | |
| 7 | Klebsiella pneumoniae | ATCC700603 | AB006 | |

### Strain Maintenance and Inoculum Preparation

Tester strains were sub-cultured on Soyabean Casein digest Agar (SCDA) and incubated at 37° C. for 18-20 hours in aerobic condition. After incubation, the isolated colonies were suspended in sterile saline to prepare the bacterial suspension. The bacterial suspension was adjusted to ˜1×10⁸ CFU/mL.

### Micro Titer Plate Preparation

[symbol] titer plates (Flat bottom) were prepared as per the CLSI recommendations. The test compound and reference compounds dilution were performed in the micro titer plates. Two hundred microliters of respective growth medium were added to the wells of a 96-wellflat bottom plate well A1 to E1 for Broth control. One hundred microliters of respective growth medium were added to all the wells B3 to B11, C3 to C11, D3 to D11, E3 to E11 except B2 to E2. One hundred microliters of prepared test compound 7 working stock solution (10 concentrations) were added from A2 to A11. Two hundred microliters of reference Standard were added to B2, C2 and D2. Two hundred microliters of DMSO were to E2 as negative control. One hundred microliter was transferred from B2, C2, D2 and E2 wells B3, C3, D3 and E3 wells respectively and serially diluted up to B11, C11, D11 and E11 wells. One hundred microliter after serial dilution was discarded from Bit, C11, D11 and E11 wells, wells A12 to F12 serve as culture control.

### Inoculation of Tester Strains

The adjusted bacterial suspension was further diluted in CAMHB (Section 6.5), 100 μl of diluted culture was added to micro titer plate where the final concentration was 5×10⁵ CFU/mL. Plates were incubated at 37° C. for 18-20 hours at aerobic conditions.

### Results and Calculation

After incubation, visual readings were recorded, and the optical density was measured at 600 nm in spectrophotometer. The concentration of compound at 90% growth was inhibited compared to growth control was considered as the MIC value. The results are presented in Table 1.

TABLE 1

| | | | MIC (μg/mL) | | | |
|---|---|---|---|---|---|---|
| S. | | | Compound | | | |
| No. | Organism | Source | 7 | Levofloxacin | Norfloxacin | Gentamicin |
| 1 | Staphylococcus aureus | ATCC25923 | 1.00 | 0.13 | 2.00 | 0.25 |
| 2 | Staphylococcus aureus | ATCC700699 | 1.00 | 16.00 | >32.00 | >32.00 |
| 3 | Staphylococcus aureus | ATCC29213 | 1.00 | 0.13 | 1.00 | 0.50 |
| 4 | Staphylococcus aureus | ATCC43300 | 1.00 | 0.13 | 1.00 | >32.00 |
| | (Gentamicin resistant) | | | | | |
| 5 | Escherichia coli | ATCC9637 | >1000 | ≤0.06 | ≤0.06 | 1.00 |
| 6 | Pseudomonas aeruginosa | ATCC 9027 | >1000 | 0.50 | 0.50 | 2.00 |
| 7 | Klebsiella pneumoniae | ATCC 700603 | >1000 | 0.50 | 4.00 | 16.00 |

ATCC 43300 is resistant to Methicillin, Oxacillin, Gentamicin and Azithromycin.
ATCC 700699 is resistant to Methicillin and Oxacillin.
ATCC25923 and ATCC29213 are sensitive strains.

As can be seen, Compound 7 is effective against various Gram-positive Staphylococcus aureus strains, including MRSA and GR-MRSA, with a MIC of 1.00 μg/mL.

1. A method of preventing or treating a condition or a disease or a disorder caused by Staphylococcus aureus (S. aureus) in a subject, comprising administering to the subject a therapeutically effective amount of a compound of Formula (I): 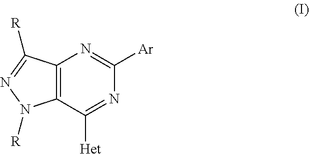
or a pharmaceutically acceptable salt, a pharmaceutically acceptable stereoisomer, a pharmaceutically acceptable hydrate, a pharmaceutically acceptable solvate, a prodrug, a polymorph, an N-oxide or a bioisostere thereof, wherein:
each R is independently selected from hydrogen, halogen, hydroxyl, optionally substituted alkyl, optionally substituted alkoxy, optionally substituted cycloalkyl, optionally substituted aryl, optionally substituted heterocyclyl, and optionally substituted heteroaryl;
Ar is an aryl or heteroaryl; and
Het represents an optionally substituted heterocyclyl or heteroaryl connected through a C—C bond.

2. The method as claimed in claim 1, wherein R is an optionally substituted alkyl.

3. The method as claimed in claim 1, wherein Ar is aryl.

4. The method as claimed in claim 3, wherein Ar is phenyl optionally substituted with one or more halo.

5. The method as claimed in claim 1, wherein Het is an optionally substituted heteroaryl.

6. The method as claimed in claim 5, wherein Het is selected from the following: 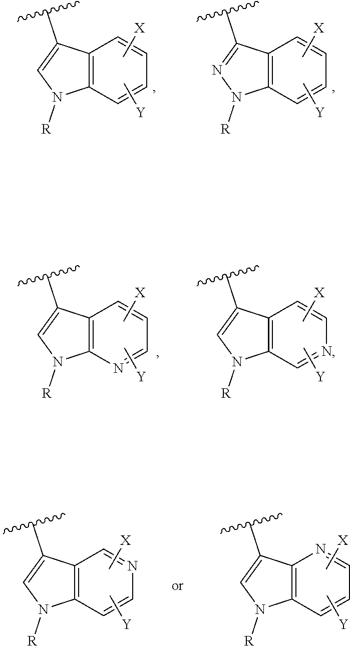
wherein,
R is independently selected from hydrogen, halogen, hydroxyl, optionally substituted alkyl, optionally substituted alkoxy, optionally substituted cycloalkyl, optionally substituted aryl, optionally substituted heterocyclyl and optionally substituted heteroaryl;
X is hydrogen or halo; and
Y is hydrogen or halo.

7. The method as claimed in claim 1, wherein the compound is selected from:
5-(4-chlorophenyl)-7-(1H-indol-3-yl)-1-methyl-3-propyl-1H-pyrazolo[4,3-d]pyrimidine;
5-(4-chlorophenyl)-1-methyl-7-(1-methyl-1H-indol-3-yl)-3-propyl-1H-pyrazolo[4,3-d]pyrimidine;
5-(4-chlorophenyl)-7-(1-ethyl-1H-indol-3-yl)-1-methyl-3-propyl-1H-pyrazolo[4,3-d]pyrimidine;
7-(5-bromo-1H-indol-3-yl)-5-(4-chlorophenyl)-1-methyl-3-propyl-1H-pyrazolo[4,3-d]pyrimidine;
7-(5-bromo-1-methyl-1H-indol-3-yl)-5-(4-chlorophenyl)-1-methyl-3-propyl-1H-pyrazolo[4,3-d]pyrimidine;
7-(5-bromo-1-ethyl-1H-indol-3-yl)-5-(4-chlorophenyl)-1-methyl-3-propyl-1H-pyrazolo[4,3-d]pyrimidine;
7-(5-chloro-1H-indol-3-yl)-5-(4-chlorophenyl)-1-methyl-3-propyl-1H-pyrazolo[4,3-d]pyrimidine;
7-(5-chloro-6-fluoro-1H-indol-3-yl)-5-(4-chlorophenyl)-1-methyl-3-propyl-1H-pyrazolo[4,3-d]pyrimidine;
5-(4-chlorophenyl)-7-(5,6-difluoro-1H-indol-3-yl)-1-methyl-3-propyl-1H-pyrazolo[4,3-d]pyrimidine;
5-(4-chlorophenyl)-7-(5-methoxy-1H-indol-3-yl)-1-methyl-3-propyl-1H-pyrazolo[4,3-d]pyrimidine;
4-chlorophenyl)-7-(5-methoxy-1-methyl-1H-indol-3-yl)-1-methyl-3-propyl-1H-pyrazolo[4,3-d]pyrimidine;
5-(4-chlorophenyl)-7-(1-ethyl-5-methoxy-1H-indol-3-yl)-1-methyl-3-propyl-1H-pyrazolo[4,3-d]pyrimidine; and
7-(1-allyl-1H-indol-3-yl)-5-(4-chlorophenyl)-1-methyl-3-propyl-1H-pyrazolo[4,3-d]pyrimidine;
or a pharmaceutically acceptable salt, a pharmaceutically acceptable stereoisomer, a pharmaceutically acceptable hydrate, a pharmaceutically acceptable solvate, a prodrug, a polymorph, an N-oxide or a bioisostere thereof.

8. The method as claimed in claim 1, wherein S. aureus is multidrug-resistant Staphylococcus aureus.

9. A method of inhibiting S. aureus comprising administering to a subject in a need thereof a therapeutically effective amount of a compound of Formula (I): 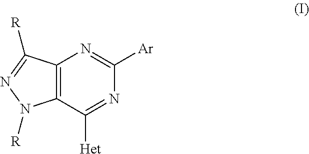
or a pharmaceutically acceptable salt, a pharmaceutically acceptable stereoisomer, a pharmaceutically acceptable hydrate, a pharmaceutically acceptable solvate, a prodrug, a polymorph, an N-oxide or a bioisostere thereof, wherein:
each R is independently selected from hydrogen, halogen, hydroxyl, optionally substituted alkyl, optionally substituted alkoxy, optionally substituted cycloalkyl, optionally substituted aryl, optionally substituted heterocyclyl, and optionally substituted heteroaryl;
Ar is an aryl or heteroaryl; and
Het represents an optionally substituted heterocyclyl or heteroaryl connected through a C—C bond.

10. The method as claimed in claim 9, wherein S. aureus is multidrug-resistant Staphylococcus aureus.

11. (canceled)

12. The method as claimed in claim 9, wherein R is an optionally substituted alkyl.

13. The method as claimed in claim 9, wherein Ar is aryl.

14. The method as claimed in claim 12 wherein Ar is phenyl optionally substituted with one or more halo.

15. The method as claimed in claim 9, wherein Het is an optionally substituted heteroaryl.

16. The method as claimed in claim 14, wherein Het is selected from the following: 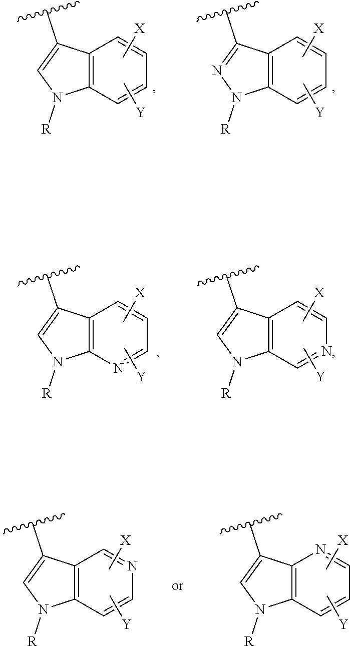
wherein,
R is independently selected from hydrogen, halogen, hydroxyl, optionally substituted alkyl, optionally substituted alkoxy, optionally substituted cycloalkyl, optionally substituted aryl, optionally substituted heterocyclyl and optionally substituted heteroaryl;
X is hydrogen or halo; and
Y is hydrogen or halo.

17. The method as claimed in claim 9, wherein the compound is selected from:
5-(4-chlorophenyl)-7-(1H-indol-3-yl)-1-methyl-3-propyl-1H-pyrazolo[4,3-d]pyrimidine;
5-(4-chlorophenyl)-1-methyl-7-(1-methyl-1H-indol-3-yl)-3-propyl-1H-pyrazolo[4,3-d]pyrimidine;
5-(4-chlorophenyl)-7-(1-ethyl-1H-indol-3-yl)-1-methyl-3-propyl-1H-pyrazolo[4,3-d]pyrimidine;
7-(5-bromo-1H-indol-3-yl)-5-(4-chlorophenyl)-1-methyl-3-propyl-1H-pyrazolo[4,3-d]pyrimidine;
7-(5-bromo-1-methyl-1H-indol-3-yl)-5-(4-chlorophenyl)-1-methyl-3-propyl-1H-pyrazolo[4,3-d]pyrimidine;
7-(5-bromo-1-ethyl-1H-indol-3-yl)-5-(4-chlorophenyl)-1-methyl-3-propyl-1H-pyrazolo[4,3-d]pyrimidine;
7-(5-chloro-1H-indol-3-yl)-5-(4-chlorophenyl)-1-methyl-3-propyl-1H-pyrazolo[4,3-d]pyrimidine;
7-(5-chloro-6-fluoro-1H-indol-3-yl)-5-(4-chlorophenyl)-1-methyl-3-propyl-1H-pyrazolo[4,3-d]pyrimidine;
5-(4-chlorophenyl)-7-(5,6-difluoro-1H-indol-3-yl)-1-methyl-3-propyl-1H-pyrazolo[4,3-d]pyrimidine;
5-(4-chlorophenyl)-7-(5-methoxy-1H-indol-3-yl)-1-methyl-3-propyl-1H-pyrazolo[4,3-d]pyrimidine;
4-chlorophenyl)-7-(5-methoxy-1-methyl-1H-indol-3-yl)-1-methyl-3-propyl-1H-pyrazolo[4,3-d]pyrimidine;
5-(4-chlorophenyl)-7-(1-ethyl-5-methoxy-1H-indol-3-yl)-1-methyl-3-propyl-1H-pyrazolo[4,3-d]pyrimidine; and
7-(1-allyl-1H-indol-3-yl)-5-(4-chlorophenyl)-1-methyl-3-propyl-1H-pyrazolo[4,3-d]pyrimidine;
or a pharmaceutically acceptable salt, a pharmaceutically acceptable stereoisomer, a pharmaceutically acceptable hydrate, a pharmaceutically acceptable solvate, a prodrug, a polymorph, an N-oxide or a bioisostere thereof.

18. (canceled)

19. (canceled)

20. (canceled)

21. The method as claimed in claim 8, wherein multidrug-resistant Staphylococcus aureus is methicillin-resistant Staphylococcus aureus (MRSA), oxacillin-resistant Staphylococcus aureus (ORSA), gentamicin-resistant Staphylococcus aureus (GRSA), or azithromycin-resistant Staphylococcus aureus (ARSA).

22. The method as claimed in claim 17, wherein multidrug-resistant Staphylococcus aureus is gentamicin-resistant Staphylococcus aureus (GRSA).

23. The method as claimed in claim 10, wherein multidrug-resistant Staphylococcus aureus is methicillin-resistant Staphylococcus aureus, oxacillin-resistant Staphylococcus aureus (ORSA), gentamicin-resistant Staphylococcus aureus (GRSA), or azithromycin-resistant Staphylococcus aureus (ARSA).

24. The method as claimed in claim 19, wherein multidrug-resistant Staphylococcus aureus is gentamicin-resistant Staphylococcus aureus (GRSA).
